(19) United States
(12) Patent Application Publication
(10) Pub. No.: US 2025/0258131 A1
(43) Pub. Date: Aug. 14, 2025

(54) BIOINFORMATICS ENABLED DEVICES, METHODS, AND SYSTEMS FOR RESOLUTION REQUIREMENT ANALYSIS

(71) Applicant: Regeneron Pharmaceuticals, Inc., Tarrytown, NY (US)

(72) Inventors: Gabriel Carreau, Delmar, NY (US); Timothy Riehlman, East Greenbush, NY (US)

(21) Appl. No.: 19/048,445

(22) Filed: Feb. 7, 2025

Related U.S. Application Data
(60) Provisional application No. 63/552,436, filed on Feb. 12, 2024.

(51) Int. Cl. G01N 27/447; G01N 33/68

(52) U.S. Cl. CPC G01N 27/44721; G01N 33/6803

(57) ABSTRACT

The present disclosure provides bioprocess devices, systems, and methods configured to analyze a sample using an analytical technique, such as electrophoresis, to characterize and quantify analytes disposed within the sample, and to perform bioinformatics operations on measurement data output from the analytical technique. Measurement data, such as relative fluorescence units (RFU) measurements over time, may be processed to determine resolution of an assay used during an analytical technique.

### CROSS REFERENCE TO RELATED APPLICATIONS

The present patent application claims priority under 35 U.S.C. § 119(e) to U.S. provisional patent application No. 63/552,436, filed Feb. 12, 2024, which application is incorporated by reference as though fully set forth herein.

### TECHNICAL FIELD

This disclosure is directed to bioinformatic devices, systems, and methods.

### INTRODUCTION

Various analytical systems and methods for analyzing biological molecules, such as proteins or nucleic acids, are known in the art. Biological molecules differ from each other in size, molecular structure, and physiochemical properties. These differences enable analysis and characterization of each molecule in a sample by separation and identification. One such analytical technique is gel electrophoresis, which includes analyzing a sample disposed in a porous medium (e.g., agarose gel or polyacrylamide gel) by separating biological molecules (e.g., proteins) based on charge, size, and molecule conformation (i.e., electrophoretic mobility). A system that conducts gel electrophoresis typically includes an assay configured to enable the characterization of specific proteins of interest in the sample. The gel electrophoresis technique may include, e.g., microchip electrophoresis coupled with laser-induced fluorescence (MCE-LIF), the output of which may include an electropherogram, which includes a data record of measurements over time, such as relative fluorescence unit (RFU) over the MCE-LIF analysis period. The data record of RFU over time is useful to determine, for example, the quantity of a protein of interest in the sample. Alternatively, the analytical technique may include capillary electrophoresis, which uses UV absorbance, and outputs a readout in absorbance units (AU). Regardless of the particular analytical technique deployed, there exists a need in the art to readily determine assay quality. The present disclosure addresses these concerns, among others, in the field of bioinformatics.

### SUMMARY OF THE DISCLOSURE

The present disclosure describes bioprocess devices, systems, and methods for bioinformatics operations. Various embodiments of the device may include one or more of the following aspects discussed herein.

In a first aspect, the disclosure provides a computer-implemented method (CIM) including: providing an electropherogram that includes relative fluorescence units (RFU) measurements over time; creating a data frame based on the electropherogram, where the data frame includes RFU measurements after a time threshold; determining a local maximum RFU of the data frame; determining a local minimum RFU of the data frame; calculating a peak-to-valley ratio based on the local maximum RFU and the local minimum RFU; and determining an assay resolution based on the peak-to-valley ratio.

In certain embodiments, the local maximum RFU includes a first peak measured after the time threshold, and the local minimum RFU includes a first valley measured after the time threshold.

In certain embodiments, the time threshold is at least 15 seconds. In certain embodiments, the time threshold is in a range between approximately 15 seconds and approximately 23 seconds.

In certain embodiments, determining the assay resolution includes comparing the peak-to-valley ratio to a resolution threshold, where the assay resolution is acceptable if the peak-to-valley ratio is greater than the resolution threshold, and where the assay resolution is not acceptable if the peak-to-valley ratio is less than the resolution threshold.

In certain embodiments, the CIM further includes creating a certificate of analysis based on the electropherogram and the assay resolution, where the certificate of analysis indicates whether the assay resolution is acceptable.

In a second aspect, the disclosure provides a computer program product (CPP), including: a machine readable storage medium; and program instructions stored on the machine readable storage medium configured to cause a processor to perform bioinformatics operations including: providing an electropherogram that includes relative fluorescence units (RFU) measurements over time; creating a data frame based on the electropherogram, where the data frame includes RFU measurements after a time threshold; determining a local maximum RFU of the data frame; determining a local minimum RFU of the data frame; calculating a peak-to-valley ratio based on the local maximum RFU and the local minimum RFU; and determining an assay resolution based on the peak-to-valley ratio.

In certain embodiments, the processor performs bioinformatics operations that further includes creating a certificate of analysis based on the electropherogram and the assay resolution, where the certificate of analysis indicates whether the assay resolution is acceptable.

In a third aspect, the disclosure provides a system including: a bioanalyzer configured to analyze a sample and to output an electropherogram that includes relative fluorescence units (RFU) measurements over time; a memory; and a processor configured to execute program instructions stored on the memory to perform a bioinformatics operation. The bioinformatics operation including: creating a data frame based on the electropherogram, where the data frame includes RFU measurements after a time threshold; determining a local maximum RFU of the data frame; determining a local minimum RFU of the data frame; calculating a peak-to-valley ratio based on the local maximum RFU and the local minimum RFU; and determining the peak-to-valley ratio which provides information on the assay's resolution.

In a fourth aspect, the disclosure provides a method including: executing an analytical technique on a sample, where the analytical technique includes microchip electrophoresis coupled with laser-induced fluorescence (MCE-LIF) configured to output an electropherogram including measurements of relative fluorescence units (RFU) over time; determining a local maximum and a local minimum of RFU measurements, including: calculating a function based on the electropherogram, wherein the function comprises a domain of timepoints after a time threshold of at least 15 seconds from a commencement of MCE-LIF, and a codomain of corresponding RFU measurements, and calculating a derivative of the function at each timepoint, where the derivative is approximately zero at the local maximum and the local minimum, and wherein the derivative transitions from positive to negative at timepoints adjacent to the local maximum, and transitions from negative to positive at timepoints adjacent to the local minimum; calculating a peak-to-valley ratio based on the local maximum and the local minimum; determining an assay resolution based on the peak-to-valley ratio; and creating a certificate of analysis that indicates whether the assay resolution is acceptable, where the assay resolution is acceptable if the peak-to-valley ratio is greater than a resolution threshold, and where the assay resolution is not acceptable if the peak-to-valley ratio is less than a resolution threshold.

In a fifth aspect, the disclosure provides a system including: a microfluidic chip configured to receive an assay and a sample therein; a bioanalyzer configured to receive the microfluidic chip therein and to execute an analytical technique on the sample, wherein the bioanalyzer comprises: a power source configured to supply electricity to the microfluidic chip for electrophoretic separation of analytes in the sample; a laser configured to induce fluorescence of analytes in the sample; a sensor configured to measure relative fluorescence units (RFU); a memory configured to store RFU measurements thereon; and a processor configured to execute program instructions stored on the memory to perform a bioinformatics operation comprising: determining a local maximum and a local minimum of RFU measured after a time threshold of at least 15 seconds, calculating a peak-to-valley ratio based on the local maximum and the local minimum, comparing the peak-to-valley ratio to a resolution threshold, and indicating whether resolution of the assay is acceptable based on the peak-to-valley ratio and the resolution threshold comparison.

In certain embodiments, the analytical technique includes microchip electrophoresis coupled with laser-induced fluorescence (MCE-LIF), and where the bioinformatics operations further includes executing MCE-LIF, which in turn outputs an electropherogram including RFU measurements over time.

In certain embodiments, the bioinformatics operation of determining the local maximum and the local minimum includes: calculating a function based on the electropherogram, where the function includes a domain of timepoints after the time threshold, and a codomain of corresponding RFU measurements; and calculating a derivative of the function at each timepoint, where the derivative is approximately zero at the local maximum and the local minimum, and where the derivative transitions from positive to negative at timepoints adjacent to the local maximum, and transitions from negative to positive at timepoints adjacent to the local minimum.

In certain embodiments, the bioinformatics operation of determining the local maximum and the local minimum includes: calculating a difference of RFU at each pair of adjacent timepoint measurements in the electropherogram after the time threshold, where the difference of RFU transitions from negative to positive at the local maximum, and transitions from positive to negative at the local minimum.

In certain embodiments, the bioinformatics operation of determining the local maximum and the local minimum includes: creating a first data structure including RFU measurements over time; in the first data structure, calculating a peak RFU that first occurs after the time threshold, where the local maximum corresponds to the peak RFU in the first data structure; creating a second data structure comprising negated RFU measurements over time; and in the second data structure, calculating a peak RFU that first occurs after the time threshold, where the local minimum corresponds to the peak RFU in the second data structure.

In certain embodiments, the assay resolution is acceptable if the peak-to-valley ratio is greater than a resolution threshold, and the assay resolution is not acceptable if the peak-to-valley ratio is less than a resolution threshold.

The terms “comprises,” “comprising,” “includes,” “including,” or any other variation thereof as used herein, are intended to cover a non-exclusive inclusion, such that a process, method, article, or apparatus that comprises a list of elements does not include only those elements, but may include other elements not expressly listed or inherent to such process, method, article, or apparatus. The term “or” is inclusive and is intended to mean that a process, method, article, or apparatus that comprises a list of elements may include a combination of or all of the elements. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items. Further, as used herein, the terms “about,” “substantially,” and “approximately” generally mean +/−10% of the indicated value.

### BRIEF DESCRIPTION OF THE FIGURES

The accompanying drawings, which are incorporated into and constitute a part of this specification, illustrate various exemplary embodiments and, together with the description, serve to explain the principles of the disclosed embodiments. The drawings show different aspects of the present disclosure and, where appropriate, reference numerals illustrating similar structures, components, materials, and/or elements in different figures are labeled similarly. It is understood that various combinations of the structures, components, and/or elements, other than those specifically shown, are contemplated and are within the scope of the present disclosure.

There are many inventions described and illustrated herein. The described inventions are neither limited to any single aspect or embodiment thereof, nor to any combinations and/or permutations of such aspects and/or embodiments. Moreover, each of the aspects of the described inventions, and/or embodiments thereof, may be employed alone or in combination with one or more of the other aspects of the described inventions and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended reflect or indicate the embodiment(s) is/are “example” embodiment(s).

FIG. 1 depicts a functional block diagram of a system according to an embodiment of the present disclosure;

FIG. 2 depicts a functional block diagram of a system according to an embodiment of the present disclosure;

FIG. 3 depicts a flow chart of a method according to an embodiment of the present disclosure;

FIG. 4 depicts a flow chart of a method according to an embodiment of the present disclosure;

FIG. 5 depicts a flow chart of a method according to an embodiment of the present disclosure; and

FIG. 6 depicts a flow chart of a method according to an embodiment of the present disclosure.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art appreciates that the side views are not drawn to scale and should not be viewed as representing proportional relationships between different components. The side views are provided to help illustrate the various components of the depicted assembly, and to show their relative positioning to one another.

### DETAILED DESCRIPTION

Embodiments of the disclosure provide a system configured to analyze samples such as, e.g., cell cultures using an analytical technique such as gel electrophoresis for protein analysis and characterization. The system may include a bioanalyzer configured to receive an assay, such as a gel assay, to characterize proteins of interest in a sample disposed within the assay. The system may be configured to output a data record, such as an electropherogram data record, associated with the protein analysis and characterization. The data record may include relative fluorescent unit (RFU) measurements over time, which may be useful to determine whether the assay provides adequate resolution according to methods of the disclosure as discussed herein.

The term “adequate resolution” as used herein refers to an assay's capability to resolve local extremum (e.g., local peak and local valley) measurements in the electropherogram data record such that ample separation is observed therebetween, and therefore the capability to provide accurate identification and quantification of local extremum. The term “local maximum” or “peak” as used herein refers to any data point whose two direct neighboring data points have a smaller amplitude in a signal. The term “local minimum” or “valley” as used herein refers to any data point whose two direct neighboring data points have a larger amplitude in a signal. The term “peak-to-valley ratio” as used herein refers to a ratio of a peak (e.g., local maximum RFU) to a valley (e.g., local minimum RFU). In some implementations, “adequate resolution” is defined as a height of a local valley which is up to ninety-nine percent (99%) of a height of a preceding local maximum, such that the peak-to-valley ratio is greater than one (1). However, “adequate resolution” criteria may vary depending on user input, minimum analysis criteria, composition of the sample, etc. For example, in other implementations, “adequate resolution” is defined as a height of a local valley between two local peaks to be no more than fifty percent (50%) of the maximum local peak height.

### I. Hardware and Software

Aspects of the present disclosure are described herein with reference to flowchart illustrations and/or block diagrams of devices, methods, systems, and computer program products according to embodiments of the disclosure. The drawings illustrate the architecture, functionality, and operation of possible implementations of devices, systems, methods, and computer program products according to various embodiments. In this regard, each block in the flowcharts or block diagrams may represent a module, segment, or portion of instructions, which includes one or more executable instructions for implementing the specified logical function(s). In alternative implementations, functions or steps noted in the block may occur out of the order shown in the drawings. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. Furthermore, each block in the drawings can be implemented by special purpose hardware-based systems that perform the specified functions or act or carry out combinations of special purpose hardware and computer instructions.

It will be understood that each block, and combination(s) of blocks, in the drawings can be implemented by computer readable program instructions. These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the drawings. These computer readable program instructions may also be stored in a computer readable storage medium that can direct a computer, a programmable data processing apparatus, and/or other device(s) to function in a particular manner, such that the computer readable storage medium having instructions stored therein comprises an article of manufacture including instructions which implement aspects of the function/act specified in the drawings. The computer readable program instructions may also be loaded onto a computer, other programmable data processing apparatus, or other device to cause a series of operational steps to be performed on the computer, other programmable apparatus, or other device to produce a computer implemented process, such that the instructions which execute on the computer, other programmable apparatus, or other device implement the functions/acts specified in the drawings.

Computer readable program instructions described herein can be downloaded to respective computing/processing devices from a computer readable storage medium or to an external computer or external storage device via a network, for example, the Internet, a local area network (LAN), a wide area network (WAN) and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may include assembler instructions, instruction-set-architecture (ISA) instructions, machine instructions, machine dependent instructions, microcode, firmware instructions, state-setting data, or either source code or object code written in any combination of at least one programming language, including an object oriented programming language such as C++, Go, Java, MatLab, Mojo, Python, R, Rust, Scala, etc., and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or a WAN, or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, to perform aspects of the disclosure. In some embodiments, computer readable program instructions are remotely stored on a cloud storage system, such that a computing device may remotely execute program instructions stored on the cloud storage system to perform aspects of the disclosure.

A computer program product, device, or system according to the present disclosure may include a computer readable storage medium (or media) having computer readable program instructions thereon for causing a processor to carry out aspects of the present disclosure. The computer readable storage medium may include a tangible device configured to receive and retain program instructions thereon for use by a computing device. The computer readable storage medium may be, for example, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device, and any suitable combination of the foregoing. In some embodiments, the computer readable storage medium may form a cloud storage system to enable remote access to data stored thereon. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

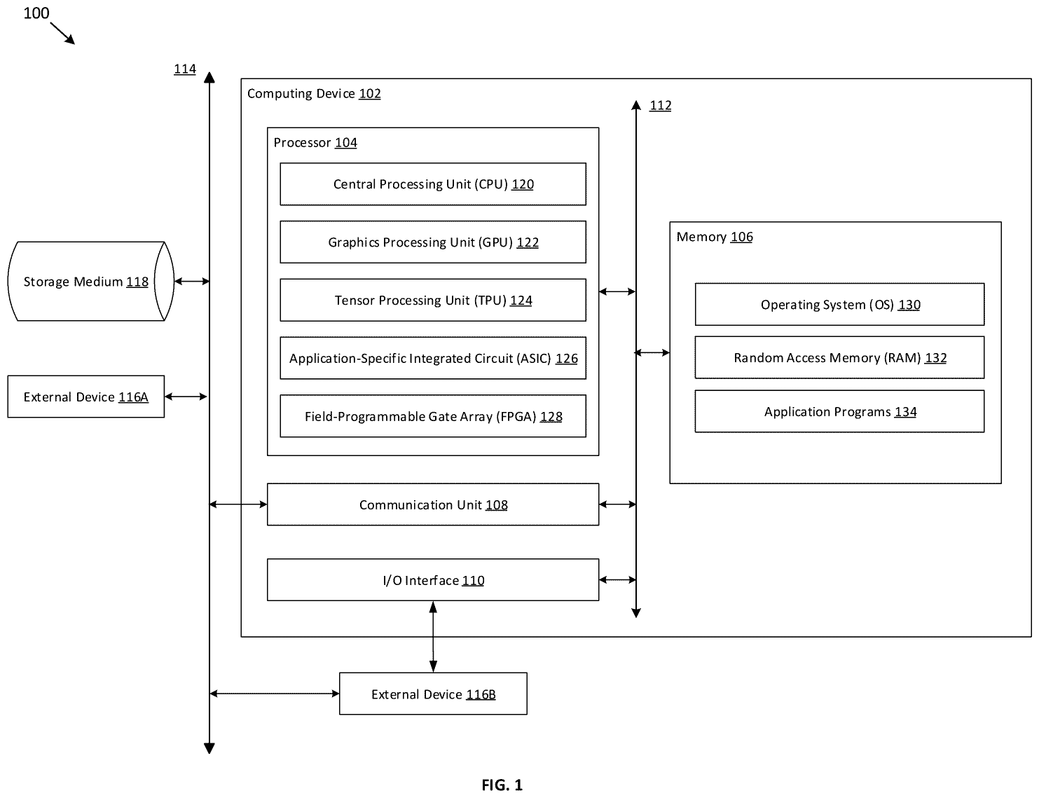

An embodiment of a possible hardware and software environment according to the present invention will now be described in detail with reference to FIG. 1.

FIG. 1 is a functional block diagram illustrating various portions of a system 100, including: a computing device 102; a processor set 104; a memory 106; a communication unit 108; an input/output (I/O) interface 110; a local interface 112; a network 114; an external device 116A; an external device 116B; and a storage medium 118. Computing device 102 may include any programmable electronic device capable of executing computer readable program instructions for carrying out operations of the present disclosure. For example, the computing device 102 may include a desktop computer, a laptop computer, a tablet computer, an integrated circuit (IC) structure, a server, a single board computer (SBC), a smart phone, etc. Computing device 102 includes various components coupled via the local interface 112, which may include any wired or wireless connections to enable appropriate communications among the various components.

Computing device 102 is capable of communicating with any number of external devices (e.g., external device 116A, external device 116B, storage medium 118, etc.), which generally includes any combination of connections and protocols that will support electrical communication. The computing device 102 may include a communication unit 108 which provides for communications with other external devices and/or systems. Communication unit 108 may provide communications over the network 114 through the use of either or both physical and wireless communications links. For example, the communication unit 108 may include a network interface card (not shown) for communicating with an external device 116A over the network 114. The external device 116A may include any device configured to communicate with the computing device 102. For example, the external device 116A may include a bioanalyzer configured to transmit analysis data to the computing device 102 for processing as discussed herein.

As further shown in FIG. 1, the computing device 102 may include an I/O interface 110 which allows for input and output of data from or to other devices that may be connected locally in data communication with the computing device 102. For example, the I/O interface 110 may include a serial port, a parallel port, an infrared (IR) interface, a radio frequency (RF) interface, a small computer system interface (SCSI), and/or a universal serial bus (USB) interface. The I/O interface 110 may be coupled with an external device 116B, which includes any electronic device configured to couple with the computing device 102 for transmitting and/or receiving data. For example, the external device 116B may include a display device, keyboard, keypad, touch screen, portable computer-readable storage media, etc. System 100 may include additional elements to enable communications, which are omitted for simplicity, such as controllers, buffers (caches), drivers, repeaters, and receivers. Computing device 102 is shown as a block diagram with double arrows representing a communications fabric, which provides communications between various components of computing device 102 and/or external devices. This communications fabric can be implemented with any architecture designed for passing data and/or controlling information between processors, system memory, peripheral devices, and any other hardware components within a system.

As further shown in FIG. 1, the computing device 102 includes a processor set 104, which may include a hardware device for executing software, including any custom made or commercially available processor configured to execute computing tasks. For example, the processor set 104 may include a central processing unit (CPU) 120, a graphics processing unit (GPU) 122, a tensor processing unit (TPU) 124, an application-specific integrated circuit (ASIC) 126, a field-programmable gate array (FPGA) 128, or a combination thereof. The processor set 104 may include multiple hardware devices configured to execute multiple computing tasks consecutively and/or in parallel. During operation of computing device 102, the processor set 104 may be configured to execute software stored within the memory 106, to communicate data to and from the various components of 102 and/or external devices 116A, 116B, and to generally control operations of the computing device 102 pursuant to the computer program instructions.

Computing device 102 further includes a memory 106, which is a computer readable storage medium, including any suitable volatile or non-volatile storage medium. Moreover, the memory 106: (i) is at least more persistent than a signal in transit; (ii) stores the program (including its soft logic and/or data), on a tangible medium (such as magnetic or optical domains); and (iii) is substantially less persistent than permanent storage. Alternatively, data storage may be more persistent and/or permanent than the type of storage provided by memory 106. In the present embodiment shown in FIG. 1, the memory 106 includes an operating system 130, a random access memory (RAM) 132, and application programs 134. The operating system 130 is configured to control the execution of other computer programs (e.g., application programs 134) and provides scheduling, input-output control, file and data management, memory management, and communication control and related services. The operating system 130 may be configured to optimize the allocation of computing resources to accomplish computing tasks, e.g., via the processor set 104. Additional elements of memory 106 enable the operation of computing device 102, such as the RAM 132 and a data cache (not shown). Application programs 134 stored on memory 106 includes computer program instructions, such as a plurality of modules, that are configured for access and/or execution by the processor set 104 to execute computing tasks according to embodiments of the disclosure. Application programs 134 may include modules to operate bioprocess equipment, or components thereof, and/or to perform bioinformatics operations discussed herein.

The programs described herein are identified based upon the application for which they are implemented in a specific embodiment of the disclosure. However, it should be appreciated that any particular program nomenclature herein is used merely for convenience, and thus the disclosure should not be limited to use solely in any specific application identified and/or implied by such nomenclature.

The descriptions of the various embodiments of the present disclosure have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

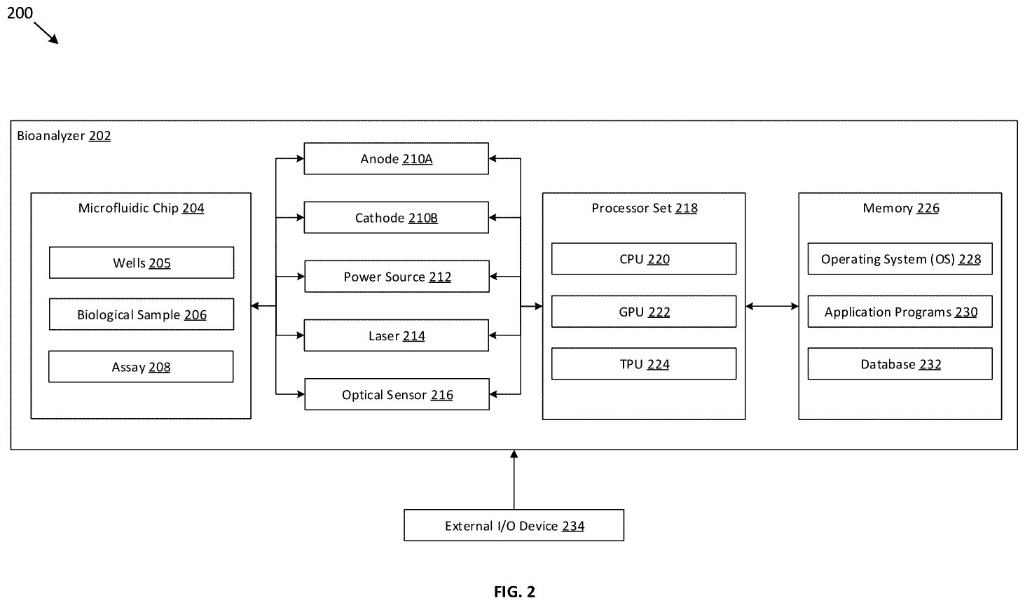

Turning to FIG. 2, illustrating a functional block diagram of an embodiment of a system 200 according to the disclosure. The system 200 includes a bioanalyzer 202 configured for characterizing and quantifying analytes in reagents or samples such as, e.g., cell culture samples disposed therein. The bioanalyzer 202 may be, e.g., a system for characterizing and performing sample quality control (QC) of biomolecules such as nucleic acids (e.g., DNA or RNA) or proteins. The bioanalyzer 202 may include, e.g., an instrument, data processing software, reagents, and a microfluidic chip specific for DNA, RNA, or protein analysis, to provide an automated electrophoresis platform for biomolecule sample quality control. Exemplary bioanalyzers include, e.g., the LabChip® GXII Touch™ Protein Characterization System by Revvity (Waltham, MA, USA), and the 2100 Bioanalyzer made by Agilent Technologies (Santa Clara, CA, USA). Other instruments may also be used to perform the functions of the bioanalyzer 202 in the context of the system 200, as will be understood by one of skill in the art. As further shown, in some implementations the system 200 further includes an external input/output (I/O) device 234, such as a keyboard device and/or display device, in communication with the bioanalyzer 202 to exchange data and/or execute bioinformatics operations discussed herein.

In the present embodiment, the bioanalyzer 202 is configured to receive a microfluidic chip 204 therein. The microfluidic chip 204 includes a well 205 that is configured to receive a sample 206 and an assay 208 therein, and thereby enable the bioanalyzer 202 to analyze the sample 206 to characterize and quantify analytes therein. Analyzing the sample 206 includes using the bioanalyzer 202 to perform an analytical technique, such as microchip electrophoresis (MCE), e.g., MCE coupled with laser-induced fluorescence (MCE-LIF). The microfluidic chip 204 is operatively coupled with an anode 210A, a cathode 210B, and a power source 212, which are collectively capable of transmitting electricity through the assay 208 and thereby electrophoretically separating analytes disposed in the sample 206. The bioanalyzer 202 further includes a laser 214 configured to induce fluorescence in analytes disposed in the sample 206, and an optical sensor 216 to measure relative fluorescent units (RFU).

In other embodiments, a bioanalyzer 202 may be used to deploy another analytical technique or detection mode used such as, e.g., capillary electrophoresis (CE). CE may use ultraviolet (UV) light absorbance to separate analytes as they pass through a narrow capillary tube, facilitating detection of compounds with chromophores that absorb UV light at specific wavelengths. The measured output may be expressed in absorbance units (AU).

The bioanalyzer 202 further includes a processor set 218, which includes any processor or combination of processors configured to execute computational tasks. In the present embodiment, the processor set 218 includes a central processing unit (CPU) 220, a graphics processing unit (222), and a tensor processing unit (TPU) 224. The processor set 218 may be configured to activate and/or control components of the bioanalyzer 202, such as the power source 212, the laser 214, and/or the optical sensor 216, according to program instructions stored in a memory 226. The memory 226 includes an operating system (OS) 228 to allocate processor resources in the processor set 218 to execute computational tasks. The memory 226 further includes application programs 230 stored thereon, which includes program instructions to operate components of the bioanalyzer 202 and to perform bioinformatics operations discussed herein. The memory 226 further includes a database 232, which provides a storage medium to organize and store data associated with the bioanalyzer 202, such as a structured query language (SQL) database to store RFU measurements output from the microfluidic chip 204.

As mentioned, in some implementations, the bioanalyzer 202 includes a LABCHIP GXII TOUCH™ protein characterization system (Revvity), however other bioanalyzer devices and/or systems are contemplated within the scope of this disclosure. In some implementations, the assay 208 is capable of analyzing protein samples having a size ranging from about 14 kilodalton (kDa) to about 200 kDa. In some implementations, the assay 208 includes a protein analysis assay, such as the LABCHIP® Pico Protein Express Assay reagent kit (Revvity), however other assays or combination of assays are contemplated within the scope of this disclosure. For example, in other implementations the assay 208 includes the LABCHIP® AAV Protein Analysis Assay (Revvity).

In some implementations, the system 200 further includes a bioreactor (not shown) configured for cultivating a sample therein under conditions which promote cell growth, protein expression, etc. The bioreactor may provide the sample 206 to the bioanalyzer 202 for analysis as discussed herein. The bioreactor may cultivate any organism (e.g., cells) capable of manufacturing biological molecules via bioprocess engineering. For example, in some implementations the sample 206 includes Chinese Hamster Ovary (CHO) cells and CHO cell lines. However other suitable cells and cell lines known in the art are contemplated within the scope of the present disclosure.

In some implementations, the bioanalyzer 202 is configured to perform a bioinformatics operation that includes determining a local maximum and a local minimum of RFU measurements from the optical sensor 216; calculating a peak-to-valley ratio based on the local maximum and local minimum RFU; and determining a resolution of the assay 208 based on the peak-to-valley ratio and a resolution threshold.

In other implementations, the bioanalyzer 202 is configured to yield an electropherogram data record having RFU measurements from the optical sensor 216, and to transmit the electropherogram data record to the computing device 102 to perform a bioinformatics operation thereon. The computing device 102 is configured to perform bioinformatics operation(s) that includes determining a local maximum and a local minimum of RFU measurements within the electropherogram data record; calculating a peak-to-valley ratio based on the local maximum and local minimum RFU; and determining a resolution of the assay 208 based on the peak-to-valley ratio and a resolution threshold.

### II. Example Embodiments

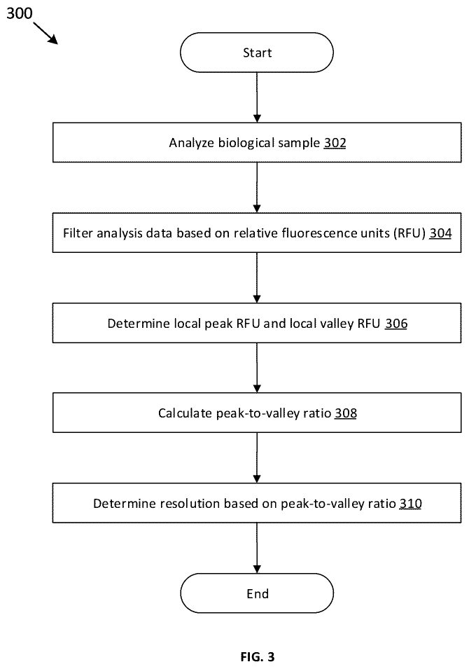

Turning now to FIG. 3, a flowchart illustrates a method 300 for a bioprocess operation according to the disclosure. Aspects of the method 300 may be implemented by devices and/or systems described herein, such as the system 100 (FIG. 1) and/or the system 200 (FIG. 2).

At step 302, the method 300 analyzes a sample using an analytical technique, or combination of analytical techniques, to characterize and quantify an analyte of interest therein. In some implementations, analyzing the sample includes electrophoretic separation of analytes in sample for characterization and quantification, for example using capillary electrophoresis (CE) and/or microchip electrophoresis (ME). In some implementations, analyzing sample includes chemiluminescence, laser-induced fluorescence, ultra-violet (UV) absorbance, electrochemical detection, and/or mass spectrometry to measure a property of analytes therein. In some implementations, analyzing the sample includes an assay, or combination of assays, to characterize and quantify analytes of interest based on the assay(s). In the present embodiment, step 302 includes microchip electrophoresis coupled with laser-induced fluorescence (MCE-LIF) to measure relative fluorescence units (RFU) of each analyte within the sample. The MCE-LIF analysis at step 302 yields an electropherogram including RFU measurements over an analysis period, such that the electropherogram includes an RFU measurement at each timepoint over the analysis period.

At step 304, the method 300 filters analysis data based on RFU measurements, and outputs a data structure having filtered RFU measurements therein. In some implementations, the data structure includes a data frame having RFU measurements and respective measurement timepoints therein. In some implementations, filtering analysis data includes extracting RFU data from the electropherogram, and removing RFU measurements that are measured before a time threshold. The time threshold may include elapsed time from a start time to a measurement time of sample analysis in step 302. In some implementations, the time threshold is at least 15 seconds. In some implementations, the time threshold ranges between approximately 15 seconds and approximately 23 seconds. In some implementations, the time threshold ranges between approximately 15 seconds and approximately 23 seconds, or between approximately 16 seconds and approximately 17 seconds.

At step 306, the method 300 determines a local peak RFU and a local valley RFU based on the data structure output at step 304. Determining the local peak RFU and the local valley RFU includes determining a local maximum RFU and a local minimum RFU, respectively, in the data structure that is measured after the time threshold. In some implementations, the local peak RFU includes the first local maximum measured after the time threshold, and the local valley RFU includes the first local minimum measured after the time threshold. In some implementations, determining the local peak RFU and local valley RFU includes using a processor to execute program instructions to parse the data structure and compare RFU measurements at adjacent timepoints. In some implementations, determining the local peak RFU and local valley RFU includes using a processor to execute program instructions to process an electropherogram image to determine local maximums and minimums therein. In some implementations, step 306 includes determining a plurality of local peaks and local valleys in the data structure.

At step 308, the method 300 calculates a peak-to-valley ratio based on the determined local peak RFU and local valley RFU output at step 306. Calculating the peak-to-valley ratio includes:

$$Ratio = \frac{Local Peak RFU}{Local Valley RFU}$$

At step 310, the method 300 determines a resolution based on the calculated peak-to-valley ratio. Step 310 includes determining whether an analytical technique, or materials used during the analytical technique, provide acceptable resolution for an intended purpose based on defined resolution criteria. In some implementations, resolution criteria include a resolution threshold which provides a minimum peak-to-valley ratio that analysis data must meet or exceed to be considered acceptable for the intended purpose. If the calculated peak-to-valley ratio is greater than the resolution threshold, then resolution is considered acceptable for the intended purpose. If the calculated peak-to-valley ratio is less than the resolution threshold, then resolution is considered not acceptable for the intended purpose. In some implementations, the resolution threshold is greater than one (1). For example, in a specific implementation, resolution threshold is 1.01. In some implementations, resolution criteria is based on the specific assay(s) used during the analytical technique. In some implementations, the resolution includes assay resolution for an assay used in the sample analysis at step 302. In some implementations, the method 300 creates a certificate of analysis specifying whether an assay is acceptable or not acceptable based on the determined resolution and resolution criteria. The certificate of analysis may include additional information about the assay, sample, analytical technique(s) and/or analytical equipment.

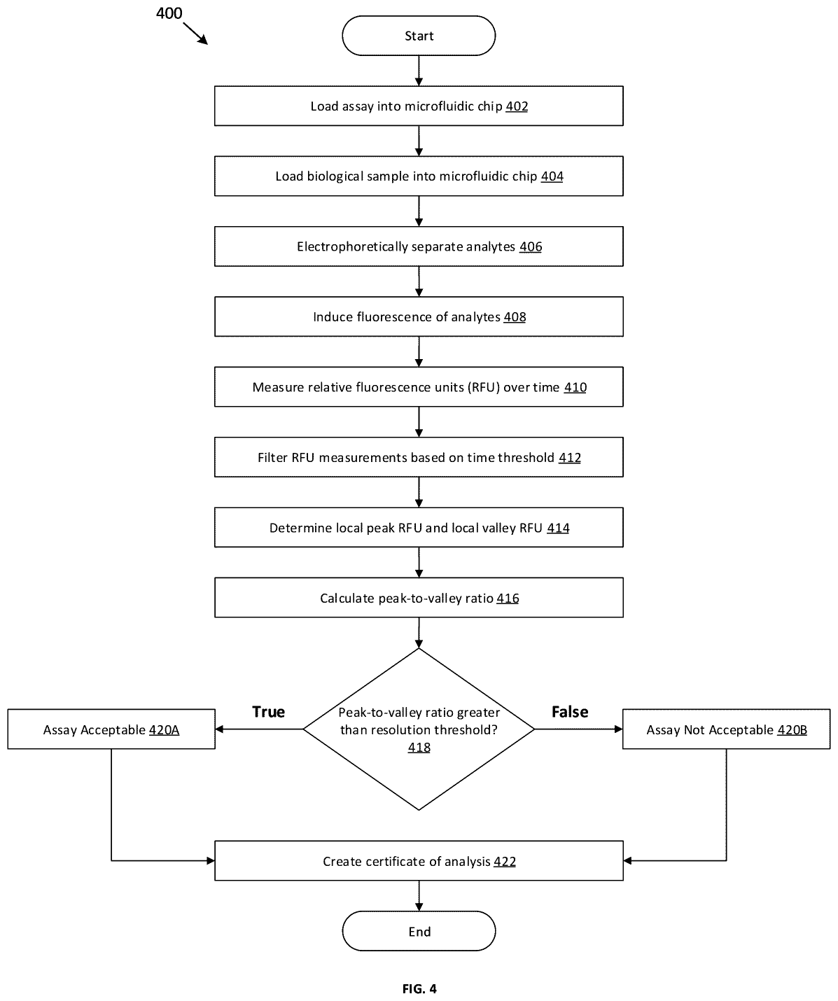

Turning now to FIG. 4, a flowchart illustrates a method 400 for a bioprocess operation according to the disclosure. Aspects of the method 400 may be implemented by devices and/or systems described herein, such as the system 100 (FIG. 1) and/or the system 200 (FIG. 2). Aspects of the method 400 discussed herein with reference to systems and methods shown in FIGS. 1-3 may be omitted for brevity.

At step 402, the method 400 loads an assay into a microfluidic chip. The microfluidic chip includes a well, or a plurality of wells, that is configured to receive the assay therein. In some implementations, loading the assay includes loading a plurality of assays into the microfluidic chip, such that each assay is loaded into at least one well therein. In some implementations, the assay includes the LABCHIP® Pico Protein Express Assay reagent kit (Revvity), however other assays or combination of assays are contemplated within the scope of this disclosure. In some implementations, the method 400 loads an assay into a plurality of microfluidic chips to analyze samples concurrently and/or consecutively.

At step 404, the method 400 loads a sample into the microfluidic chip. In some implementations, loading the sample includes aspirating a sample, such as a cell culture supernatant having an expressed protein therein, into a passage within the microfluidic chip and/or onto a surface of the microfluidic chip. In some implementations, loading the sample includes aspirating a plurality of samples into a plurality of passages (e.g., wells) within the microfluidic chip and/or onto a plurality of surfaces of the microfluidic chip.

At step 406, the method 400 electrophoretically separates analytes in the sample. The bioanalyzer includes electrodes that provide voltage and current control to enable electrophoretic separation within the microfluidic chip. Separating analytes in the sample includes loading the microfluidic chip into a bioanalyzer configured to execute an electrophoresis technique, such as MCE-LIF. In some implementations, the bioanalyzer includes a LABCHIPx GXII TOUCH (Revvity), however other bioanalyzer devices and/or systems are contemplated within the scope of this disclosure. At step 408, the method 400 induces fluorescence of each analyte within the microfluidic chip using a laser. In some implementations, inducing fluorescence includes labeling the sample with a dye prior to analysis, such as lyophilized labeling dye of the LABCHIP® Pico Protein Express Assay reagent kit (Revvity), which emits an excitation signal in response to electromagnetic exposure. At step 410, the method 400 measures RFU over time which corresponds to RFU measurements for each analyte. The bioanalyzer in the present embodiment is therefore capable of performing MCE-LIF to characterize and quantify analytes of interest.

At step 412, the method 400 filters RFU measurements based on a time threshold. In the present embodiment, filtering RFU measurements includes removing data points measured before a time threshold of at least 15 seconds. The method 400 then determines a local peak RFU and a local valley RFU at step 414, and in turn calculates a peak-to-valley ratio based on the local peak and the local valley at step 416.

At step 418, the method 400 determines whether the calculated peak-to-valley ratio is greater than a resolution threshold. If the peak-to-valley ratio is greater than the resolution threshold, then the assay is determined to be acceptable at step 420A. If the peak-to-valley ratio is less than the resolution threshold, then the assay is determined to be unacceptable at step 420B. After the determination is made at step 420A or step 420B, the method 400 proceeds to create a certificate of analysis at step 422. The certificate analysis includes an indication of whether the assay is acceptable or not acceptable based on the determination at step 420A or step 420B, respectively. The certificate of analysis may further include additional information about the analysis, such as characterization and/or quantification information about analytes in sample.

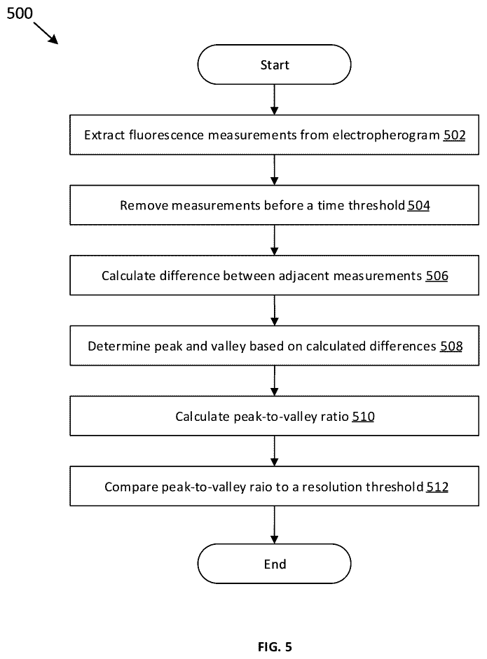

Turning now to FIG. 5, a flowchart illustrates a method 500 for a bioinformatics operation according to the disclosure. Aspects of the method 500 may be implemented by devices and/or systems described herein, such as the system 100 (FIG. 1) and/or the system 200 (FIG. 2). Aspects of the method 500 discussed herein with reference to systems and methods shown in FIGS. 1-4 may be omitted for brevity.

At step 502, the method 500 extracts fluorescence measurements from an electropherogram. Fluorescence measurements includes relative fluorescence units (RFU) measurements at each timepoint over an analysis period. In some implementations, a bioanalyzer outputs the electropherogram after analyzing a sample using an electrophoresis technique, as discussed elsewhere herein.

At step 504, the method 500 removes RFU measurements that occur before a time threshold. In some implementations, this includes removing RFU measurements that occur before the first 15 seconds of analyzing the sample. In other implementations, this includes removing RFU measurements that occur before the first 23 seconds of analyzing the sample. In a specific implementation, this includes removing RFU measurements that occur before the first 16, 17, 18, 19, 20, 21, 22, or 23 seconds of analyzing the sample. In some implementations, the method 500 creates a data frame having fluorescence measurements and corresponding time points that occur after the time threshold.

At step 506, the method 500 calculates the difference between adjacent RFU measurements after the time threshold, and stores calculated differences in a data structure. For instance, step 506 may calculate a difference (Dt) between a first measurement at a first time point, RFU (t), and a second measurement at a second, subsequent time point, RFU (t+1), for each timepoint after the threshold, and in turn store calculated differences in an array as shown below:

$${D}_{t} = RFU ( t ) - RFU ( t + 1 )$$

$$Array = \{ {D}_{15} , {D}_{16} , {D}_{17} … {D}_{n} \}$$

At step 508, the method 500 determines a peak and valley based on the calculated differences output at step 506. RFU measurements are increasing if the calculated difference is negative, and RFU measurements are decreasing if the calculated difference is positive. This enables the method 500 to parse the calculated differences to determine the peaks and valleys based on the calculated differences of adjacent measurements. A local peak can be identified at an inflection point where the calculated differences transition from negative to positive. A local valley can be identified at an inflection point where the calculated differences transition from positive to negative. The method 500 then calculates a peak-to-valley ratio at step 510 based on the determined peak and valley, and in turn compares the calculated peak-to-valley ratio to a resolution threshold at 512 as previously discussed herein.

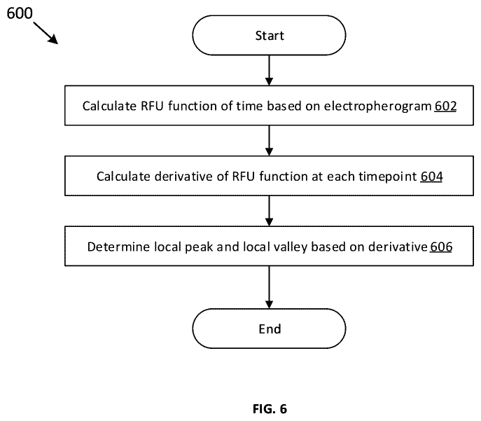

Turning now to FIG. 6, a flowchart illustrates a method 600 for a bioinformatics operation according to the disclosure. Aspects of the method 600 may be implemented by devices and/or systems described herein, such as the system 100 (FIG. 1) and/or the system 200 (FIG. 2). Aspects of the method 600 discussed herein with reference to systems and methods shown in FIGS. 1-5 may be omitted for brevity.

At step 602, the method 600 calculates an RFU function of time based on an electropherogram including RFU measurements therein. The calculated RFU function includes a domain of timepoints and codomain of RFU measurements, and therefore can be used to calculate a respective RFU measurement at a respective timepoint. Calculating the RFU function may include known mathematical processes, omitted for brevity, for calculating functions based on a series of data points.

At step 604, the method 600 calculates a derivative of the RFU function, and determines the derivative value at each timepoint. In some implementations, step 604 only determines the derivative at each timepoint after a time threshold, such as time threshold in a range between 15 seconds and 30 seconds, between 15 seconds and 25 seconds, between 15 seconds and 23 seconds, or between 15 seconds and 20 seconds, e.g., 15 seconds, 16 seconds, 17 seconds, 18 seconds, 19 seconds, 20 seconds, 21 seconds, 22 seconds, 23 seconds, 24 seconds, 25 seconds, 26 seconds, 27 seconds, 28 seconds, 29 seconds, or 30 seconds after RFU measurements in the electropherogram begin. The method 600 may store the calculated derivatives in a data structure for further processing. Example calculations may include:

Derivative=RFU′(t)

Array Derivative={RFU′(15),RFU′(16),RFU′(17) . . . RFU′(n)}

At step 606, the method 600 determines a local peak and a local valley based on the calculated derivatives. The calculated derivative value at each timepoint corresponds to the slope of the RFU function at the respective timepoint. The slope at each timepoint, and slope of neighboring timepoints, may be used to determine local peaks and local valleys. At a local peak or a local valley of the RFU function, the slope is approximately equal to zero. The RFU function has a positive slope as it approaches a local peak, has a slope of approximately zero at the local peak, and then a negative slope as it moves away from the local peak. The RFU function has a negative slope as it approaches a local valley, has a slope of approximately zero at the local valley, and then a positive slope as it moves away from the local valley. The transition from positive to negative slope and from negative to positive slope are useful to respectfully determine the local peak and local valley. The method 600 may then use the determined local peak and local valley to calculate a peak-to-valley ratio to perform other bioinformatics operations as discussed elsewhere herein.

### III. Items

Embodiments of the present disclosure may include the following features:
Item 1. A computer-implemented method (CIM) comprising: providing an electropherogram that comprises relative fluorescence units (RFU) measurements over time; creating a data frame based on the electropherogram, wherein the data frame comprises RFU measurements after a time threshold; determining a local maximum RFU of the data frame; determining a local minimum RFU of the data frame; calculating a peak-to-valley ratio based on the local maximum RFU and the local minimum RFU; and determining an assay resolution based on the peak-to-valley ratio.
Item 2. The CIM of item 1, wherein the local maximum RFU comprises a first peak measured after the time threshold, and wherein the local minimum RFU comprises a first valley measured after the time threshold.
Item 3. The CIM of item 1, wherein the time threshold is at least 15 seconds.
Item 4. The CIM of item 1, wherein the time threshold is in a range between approximately 15 seconds and approximately 23 seconds.
Item 5. The CIM of item 1, wherein determining the assay resolution comprises comparing the peak-to-valley ratio to a resolution threshold, wherein the assay resolution is acceptable if the peak-to-valley ratio is greater than the resolution threshold, and wherein the assay resolution is not acceptable if the peak-to-valley ratio is less than the resolution threshold.
Item 6. The CIM of item 5, further comprising: creating a certificate of analysis based on the electropherogram and the assay resolution, wherein the certificate of analysis indicates whether the assay resolution is acceptable.
Item 7. A computer program product (CPP), comprising: a machine readable storage medium; and program instructions stored on the machine readable storage medium configured to cause a processor to perform bioinformatics operations comprising: providing an electropherogram that comprises relative fluorescence units (RFU) measurements over time; creating a data frame based on the electropherogram, wherein the data frame comprises RFU measurements after a time threshold; determining a local maximum RFU of the data frame; determining a local minimum RFU of the data frame; calculating a peak-to-valley ratio based on the local maximum RFU and the local minimum RFU; and determining an assay resolution based on the peak-to-valley ratio.
Item 8. The CPP of item 7, wherein the local maximum RFU comprises a first peak measured after the time threshold, and wherein the local minimum RFU comprises a first valley measured after the time threshold.
Item 9. The CPP of item 7, wherein the time threshold is at least 15 seconds.
Item 10. The CPP of item 7, wherein the time threshold is in a range between approximately 15 seconds and approximately 23 seconds.
Item 11. The CPP of item 7, wherein determining the assay resolution comprises comparing the peak-to-valley ratio to a resolution threshold, wherein the assay resolution is acceptable if the peak-to-valley ratio is greater than the resolution threshold, and wherein the assay resolution is not acceptable if the peak-to-valley ratio is less than the resolution threshold.
Item 12. The CPP of item 11, wherein the processor performs bioinformatics operations further comprising: creating a certificate of analysis based on the electropherogram and the assay resolution, wherein the certificate of analysis indicates whether the assay resolution is acceptable.
Item 13. A system comprising: a bioanalyzer configured to analyze a sample and to output an electropherogram that comprises relative fluorescence units (RFU) measurements over time; a memory; a processor configured to execute program instructions stored on the memory to perform a bioinformatics operation comprising: creating a data frame based on the electropherogram, wherein the data frame comprises RFU measurements after a time threshold; determining a local maximum RFU of the data frame; determining a local minimum RFU of the data frame; calculating a peak-to-valley ratio based on the local maximum RFU and the local minimum RFU; and determining an assay resolution based on the peak-to-valley ratio.
Item 14. The system of item 13, wherein the local maximum RFU comprises a first peak measured after the time threshold, and wherein the local minimum RFU comprises a first valley measured after the time threshold.
Item 15. The system of item 13, wherein the time threshold is at least 15 seconds.
Item 16. The system of item 13, wherein the time threshold is in a range between approximately 15 seconds and approximately 23 seconds.
Item 17. The system of item 13, wherein determining the assay resolution comprises: comparing the peak-to-valley ratio to a resolution threshold, wherein the assay resolution is acceptable if the peak-to-valley ratio is greater than the resolution threshold, and wherein the assay resolution is not acceptable if the peak-to-valley ratio is less than the resolution threshold.
Item 18. The system of item 17, wherein the processor performs bioinformatics operations further comprising: creating a certificate of analysis based on the electropherogram and the assay resolution, wherein the certificate of analysis indicates whether the assay resolution is acceptable.
Item 19. A method comprising: executing an analytical technique on a sample, wherein the analytical technique comprises microchip electrophoresis coupled with laser-induced fluorescence (MCE-LIF) configured to output an electropherogram comprising measurements of relative fluorescence units (RFU) over time; determining a local maximum and a local minimum of RFU measurements, comprising: calculating a function based on the electropherogram, wherein the function comprises a domain of timepoints after a time threshold of at least 15 seconds from a commencement of MCE-LIF, and a codomain of corresponding RFU measurements, and calculating a derivative of the function at each timepoint, wherein the derivative is approximately zero at the local maximum and the local minimum, and wherein the derivative transitions from positive to negative at timepoints adjacent to the local maximum, and transitions from negative to positive at timepoints adjacent to the local minimum; calculating a peak-to-valley ratio based on the local maximum and the local minimum; determining an assay resolution based on the peak-to-valley ratio; and creating a certificate of analysis that indicates whether the assay resolution is acceptable, wherein the assay resolution is acceptable if the peak-to-valley ratio is greater than a resolution threshold, and wherein the assay resolution is not acceptable if the peak-to-valley ratio is less than a resolution threshold.
Item 20. A system comprising: a microfluidic chip configured to receive an assay and a sample therein; a bioanalyzer configured to receive the microfluidic chip therein and to execute an analytical technique on the sample, wherein the bioanalyzer comprises: a power source configured to supply electricity to the microfluidic chip for electrophoretic separation of analytes in the sample; a laser configured to induce fluorescence of analytes in the sample; a sensor configured to measure relative fluorescence units (RFU); a memory configured to store RFU measurements thereon; and a processor configured to execute program instructions stored on the memory to perform a bioinformatics operation comprising: determining a local maximum and a local minimum of RFU measured after a time threshold of at least 15 seconds, calculating a peak-to-valley ratio based on the local maximum and the local minimum, comparing the peak-to-valley ratio to a resolution threshold, and indicating whether resolution of the assay is acceptable based on the peak-to-valley ratio and the resolution threshold comparison.
Item 21. The system of item 20, wherein the analytical technique comprises microchip electrophoresis coupled with laser-induced fluorescence (MCE-LIF), and wherein the bioinformatics operations further comprise executing MCE-LIF, which in turn outputs an electropherogram comprising RFU measurements over time.
Item 22. The system of item 21, wherein the bioinformatics operation of determining the local maximum and the local minimum comprises: calculating a function based on the electropherogram, wherein the function comprises a domain of timepoints after the time threshold, and a codomain of corresponding RFU measurements; and calculating a derivative of the function at each timepoint, wherein the derivative is approximately zero at the local maximum and the local minimum, and wherein the derivative transitions from positive to negative at timepoints adjacent to the local maximum, and transitions from negative to positive at timepoints adjacent to the local minimum.
Item 23. The system of item 21, wherein the bioinformatics operation of determining the local maximum and the local minimum comprises: calculating a difference of RFU at each pair of adjacent timepoint measurements in the electropherogram after the time threshold, wherein the difference of RFU transitions from negative to positive at the local maximum, and transitions from positive to negative at the local minimum.
Item 24. The system of item 21, wherein the bioinformatics operation of determining the local maximum and the local minimum comprises: creating a first data structure comprising RFU measurements over time; in the first data structure, calculating a peak RFU that first occurs after the time threshold, wherein the local maximum corresponds to the peak RFU in the first data structure; creating a second data structure comprising negated RFU measurements over time; and in the second data structure, calculating a peak RFU that first occurs after the time threshold, wherein the local minimum corresponds to the peak RFU in the second data structure.
Item 25. The system of item 20, wherein the assay resolution is acceptable if the peak-to-valley ratio is greater than a resolution threshold, and the assay resolution is not acceptable if the peak-to-valley ratio is less than a resolution threshold.

What is claimed is:

1. A computer-implemented method (CIM) comprising:
providing an electropherogram that comprises relative fluorescence units (RFU) measurements over time;
creating a data frame based on the electropherogram, wherein the data frame comprises RFU measurements after a time threshold;
determining a local maximum RFU of the data frame;
determining a local minimum RFU of the data frame;
calculating a peak-to-valley ratio based on the local maximum RFU and the local minimum RFU; and
determining an assay resolution based on the peak-to-valley ratio.

2. The CIM of claim 1, wherein the local maximum RFU comprises a first peak measured after the time threshold, and wherein the local minimum RFU comprises a first valley measured after the time threshold.

3. The CIM of claim 1, wherein the time threshold is at least 15 seconds.

4. The CIM of claim 1, wherein the time threshold is in a range between approximately 15 seconds and approximately 23 seconds.

5. The CIM of claim 1, wherein determining the assay resolution comprises comparing the peak-to-valley ratio to a resolution threshold,
wherein the assay resolution is acceptable if the peak-to-valley ratio is greater than the resolution threshold, and
wherein the assay resolution is not acceptable if the peak-to-valley ratio is less than the resolution threshold.

6. The CIM of claim 5, further comprising:
creating a certificate of analysis based on the electropherogram and the assay resolution, wherein the certificate of analysis indicates whether the assay resolution is acceptable.

7. A computer program product (CPP), comprising:
a machine readable storage medium; and
program instructions stored on the machine readable storage medium configured to cause a processor to perform bioinformatics operations comprising:
providing an electropherogram that comprises relative fluorescence units (RFU) measurements over time;
creating a data frame based on the electropherogram, wherein the data frame comprises RFU measurements after a time threshold;
determining a local maximum RFU of the data frame;
determining a local minimum RFU of the data frame;
calculating a peak-to-valley ratio based on the local maximum RFU and the local minimum RFU; and
determining an assay resolution based on the peak-to-valley ratio.

8. The CPP of claim 7, wherein the local maximum RFU comprises a first peak measured after the time threshold, and wherein the local minimum RFU comprises a first valley measured after the time threshold.

9. The CPP of claim 7, wherein the time threshold is at least 15 seconds.

10. The CPP of claim 7, wherein the time threshold is in a range between approximately 15 seconds and approximately 23 seconds.

11. The CPP of claim 7, wherein determining the assay resolution comprises comparing the peak-to-valley ratio to a resolution threshold,
wherein the assay resolution is acceptable if the peak-to-valley ratio is greater than the resolution threshold, and
wherein the assay resolution is not acceptable if the peak-to-valley ratio is less than the resolution threshold.

12. The CPP of claim 11, wherein the processor performs bioinformatics operations further comprising:
creating a certificate of analysis based on the electropherogram and the assay resolution, wherein the certificate of analysis indicates whether the assay resolution is acceptable.

13. A system comprising:
a bioanalyzer configured to analyze a sample and to output an electropherogram that comprises relative fluorescence units (RFU) measurements over time;
a memory; and
a processor configured to execute program instructions stored on the memory to perform a bioinformatics operation comprising:
creating a data frame based on the electropherogram, wherein the data frame comprises RFU measurements after a time threshold;
determining a local maximum RFU of the data frame;
determining a local minimum RFU of the data frame;
calculating a peak-to-valley ratio based on the local maximum RFU and the local minimum RFU; and
determining an assay resolution based on the peak-to-valley ratio.

14. The system of claim 13, wherein the local maximum RFU comprises a first peak measured after the time threshold, and wherein the local minimum RFU comprises a first valley measured after the time threshold.

15. The system of claim 13, wherein the time threshold is at least 15 seconds.

16. The system of claim 13, wherein the time threshold is in a range between approximately 15 seconds and approximately 23 seconds.

17. The system of claim 13, wherein determining the assay resolution comprises:
comparing the peak-to-valley ratio to a resolution threshold,
wherein the assay resolution is acceptable if the peak-to-valley ratio is greater than the resolution threshold, and
wherein the assay resolution is not acceptable if the peak-to-valley ratio is less than the resolution threshold.

18. The system of claim 17, wherein the processor performs bioinformatics operations further comprising:
creating a certificate of analysis based on the electropherogram and the assay resolution, wherein the certificate of analysis indicates whether the assay resolution is acceptable.
